(19) United States
(12) Patent Application Publication
(10) Pub. No.: US 2009/0299370 A1
(43) Pub. Date: Dec. 3, 2009

(54) DYNAMIZATION OF FIXED SCREW FRACTURE PLATES

(76) Inventor: P. Douglas Kiester, Irvine, CA (US)

Correspondence Address: P. DOUGLAS KIESTER;CBRK ENTERPRISES, 19 Rippling Stream, Irvine, CA, 92603, US

(21) Appl. No.: 12/129,563

(22) Filed: May 29, 2008

(51) Int. Cl. A61B 17/56; A61B 17/80

(52) U.S. Cl. 606/71; 606/280; 606/70

(57) ABSTRACT

A locking fracture plate may become a dynamic locking plate with the removal of a single set screw. The set screw removal may be done at the time of the initial implantation of the fracture plate, or several weeks later after the fracture has been allowed to consolidate. The device of the present invention may allow for the set screw to be removed percutaneously under x-ray control. The dynamized plate may allow for fracture compression but not for fracture displacement (loss or reduction) by rotation or sheer. The dynamized plate, with the set screw removed, may still protect the fracture from rotation and sheer. While conventional fracture plates may rigidly hold the bones in place, thereby potentially slowing bone fusion, or, in extreme cases, resulting in nonunion of the fracture, the fracture plate of the present invention may allow for fracture compression and, therefore, permit bone healing, growth and repair.

### BACKGROUND OF THE INVENTION

The present invention relates to apparatus and methods for fixation of fractured bones and, more particularly, to the dynamization of fixed screw fracture plates, allowing for fracture compression without fracture displacement by rotation or sheer.

External bone fixators were developed to enable surgeons to reestablish the alignment of bone pieces at a fracture site, and to reduce and stabilize the fracture to promote healing. Such fixators generally attach to the bone on opposite sides of the fracture.

The introduction of fixed plates for the treatment of fractures is one of the most significant advances for the surgical treatment of fractures in recent history. Conventional screws apply pressure to the plate as they are tightened, which holds the plate to the bone, but do not attach directly to the plate. More recent technology consists of screws whose heads screw into the plates so that the screws are firmly fixed to the plate as they are inserted. This more recent configuration greatly adds to the rigidity of the plate, screw, bone construct.

Because the plates no longer need to be squeezed tightly against the bone to gain stability, the plates can stand-off a little from the bone and still give the fracture rigid stability. In theory this helps the blood supply to get back into the fracture area to aid in healing. Because the plate is not firmly against the bone, these are often called internal-external fixators.

In addition, with this more recent configuration, with the screw heads screwing into the plates, all the screws are locked. If the screws pull out or fail, all of the screws must fail simultaneously, rather than one at a time, as in a standard screw-plate construct. For old, osteopenic, or soft bone fractures, this is a major advance.

Now that bone fracture fixed plates have been in use for several years, their biggest weakness has become apparent. External fixators are notoriously slow to allow the bone to heal. One major theory why is that they are too rigid and don't allow the fracture to compress. The more recent configuration described above often show the same tendency of delayed healing, again probably because they are too rigid and don't allow the fracture to compress.

As can be seen, there is a need for a bone fixation device and method that may allow for fracture compression while still maintaining the reduction and stabilization of the fracture.

### SUMMARY OF THE INVENTION

In one aspect of the present invention, a fracture plate comprises a fracture plate body having at least one screw hole therethrough; a slot formed in the fracture plate body; a sliding bar slidably disposed in the slot, the sliding bar having at least one screw hole therethrough; and a set screw threadably disposed in the fracture plate, wherein the set screw fixes the sliding bar in a first position when the set screw is inserted in the fracture plate; and the removal of the set screw permits the sliding bar to slide in the slot of the fracture plate.

In another aspect of the present invention, a fracture fixation kit comprises a fracture plate comprising a fracture plate body with at least one screw hole therethrough; a slot formed in the fracture plate body; a sliding bar slidably disposed in the slot, the sliding bar having at least one screw hole therethrough; and a set screw threadably disposed in the fracture plate; and a screw extractor.

In a further aspect of the present invention, a method for fixing a fractured bone comprises attaching one end of a fracture plate body of a fracture plate to a bone at one side of a bone fracture, the fracture plate comprising a slot formed in the fracture plate body at another end of the fracture plate body; a sliding bar slidably disposed in the slot, the sliding bar having at least one screw hole therethrough; and a set screw threadably disposed in the fracture plate, the set screw holding the sliding bar at a first position in the slot; attaching the sliding bar to another side of the bone fracture, wherein the fracture plate is attached to the bone on both sides of the bone fracture; and removing the set screw to permit compression of the bone fracture.

These and other features, aspects and advantages of the present invention will become better understood with reference to the following drawings, description and claims.

### BRIEF DESCRIPTION OF THE DRAWINGS

FIG. 1 is a perspective front view of the fracture plate of the present invention;

FIG. 2 is a perspective back view of the fracture plate of FIG. 1;

FIG. 3 is a cross-sectional view of the fracture plate taken along line 3-3 of FIG. 1;

FIG. 4A is a perspective front view of the fracture plate of FIG. 1 with the locking screw removed according to the present invention;

FIG. 4B is a perspective front view of the fracture plate of FIG. 4A showing the ability of the sliding bar to move according to the present invention;

FIG. 5A is a perspective view of a locking screw according to the present invention;

FIG. 5B is a side view of the locking screw of FIG. 5A;

FIG. 5C is a plan view of the locking screw of FIG. 5A;

FIG. 6 is a plan close-up view of the fracture plate of the present invention with the locking screw in place according to the present invention;

FIG. 7A is a perspective view of a locking screw extractor according to the present invention;

FIG. 7B is a close-up view of the tip of the locking screw extractor of FIG. 7A;

FIG. 8A is a perspective view of the screw extractor of FIG. 7A being inserted into the locking screw of the present invention;

FIG. 8B is a side close-up view of the screw extractor of FIG. 7A engaging the locking screw;

FIG. 8C is a close-up view of the engagement of the screw extractor of FIG. 7A engaging the locking screw;

FIG. 9 is a perspective view of a scar extractor useable with the fracture plate of the present invention;

FIG. 10A is a perspective view of the insertion of a working tube inserter according to the present invention;

FIG. 10B is perspective view of the working tube inserter of FIG. 10A fully inserted to the fracture plate;

FIG. 10C is a perspective view showing a working tube being inserted over the working tube inserter according to the present invention;

FIG. 10D is a perspective view showing the working tube fully inserted to the fracture plate;

FIG. 11 is a perspective view of the screw extractor of FIG. 7A being inserted through the working tube according to the present invention; and

FIG. 12 is a flow chart describing a method for securing a bone fracture according to the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides apparatus and methods for the dynamization of fixed screw fracture plates. The device of the present invention may be a locking fracture plate which may become a dynamic locking plate with the removal of a single locking screw (also referred to as a set screw). The set screw removal may be done at the time of the initial implantation, or several weeks later after the fracture has been allowed to consolidate. The device of the present invention may allow for the set screw to be removed percutaneously under x-ray control. The dynamized plate may allow for fracture compression but not for fracture displacement (loss or reduction) by rotation or sheer. The dynamized plate, with the set screw removed, may still protect the fracture from rotation and sheer.

Conventional fracture plates may rigidly hold the bones in place, thereby potentially slowing bone fusion, or, in extreme cases, resulting in nonunion of the fracture. The fracture plate of the present invention may allow for fracture compression and, therefore, permit bone healing, growth and repair.

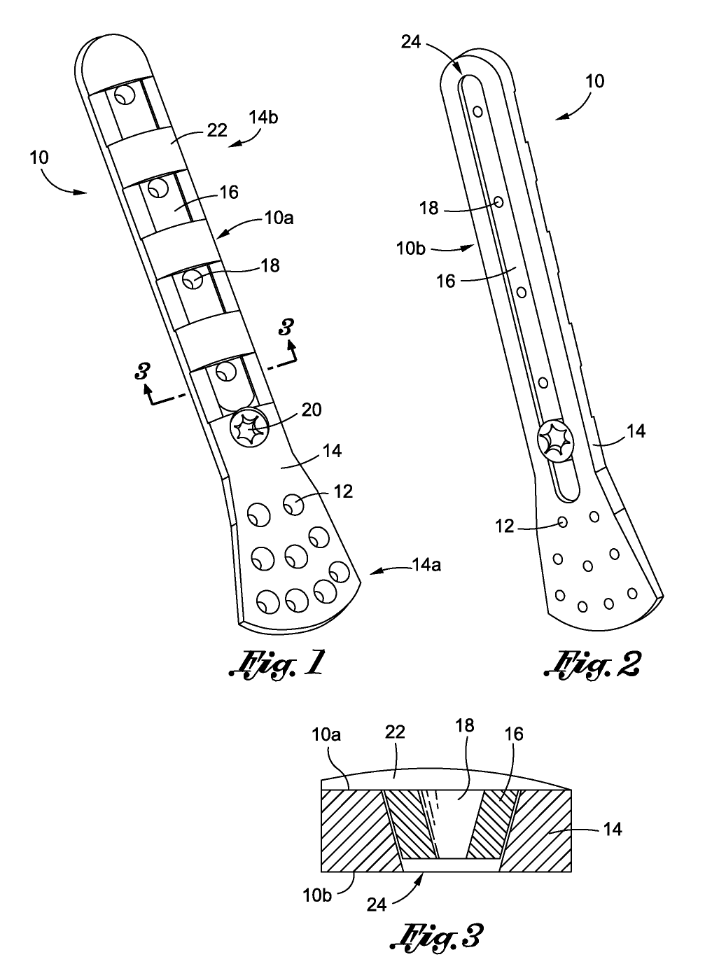

Referring to FIGS. 1 and 2, there is shown perspective views of the front 10a and back 10b, respectively, of a fracture plate 10 according to the present invention. The fracture plate 10 may have a plurality of screw holes 12 for securing the fracture plate body 14 to a bone at one end (e.g., distal or proximal) of a bone fracture. The fracture plate 10 may include a sliding bar 16 which may slidably move within a slot 24 in the fracture plate body 14. The screw holes 12 may be formed on one end 14a of the fracture plate body 14 and the slot 24 may be formed at a second, opposite end 14b of the fracture plate body 14. The sliding bar 16 may include a plurality of screw holes 18 for securing the sliding bar 16 to a bone at another end of a bone fracture.

The sliding bar 16 may be held in a first position, as shown in FIGS. 1 and 2, with a locking or set screw 20. In this position, the screw holes 12 of the fracture plate body 14 are a maximum distance from the screw holes 18 of the sliding bar 16. When the set screw 20 is removed, the sliding bar 16 may be permitted to slide in the slot 24. The slot 24 may extend beyond the set screw 24, as shown in FIG. 2, thereby allowing the sliding bar 16 to slide a distance greater than the diameter of the set screw 20 when the set screw 20 is removed.

The fracture plate 10 may include a plurality of cross links 22. The cross links 22 may hold the sliding bar 16 within a slot 24 formed in the fracture plate body 14. The cross links 22 may be formed on the front 10a of the fracture plate 10 as shown in FIG. 1. Optionally, the cross links 22 may be formed on the bottom 10b of the fracture plate 10.

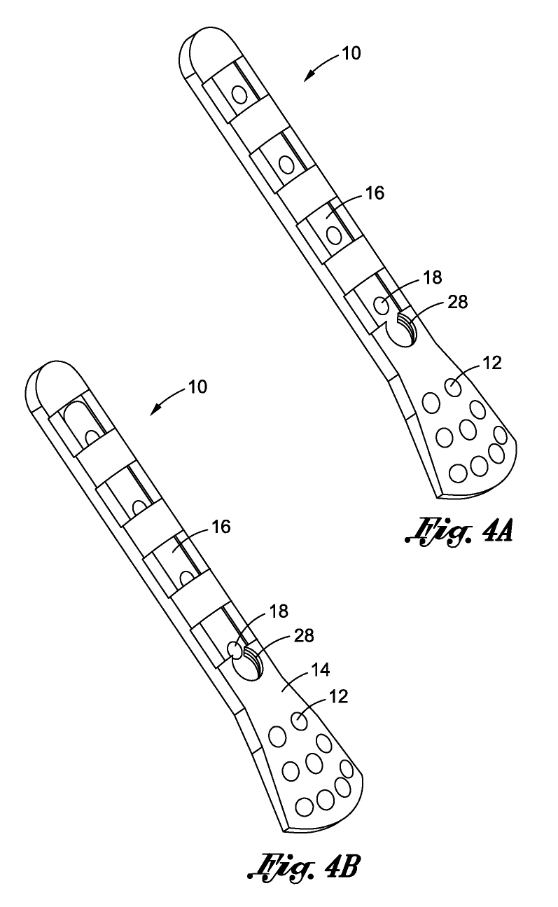

In the absence of cross links 22 on the bottom 10b of the fracture plate 10, the slot 24 may have a V-shape as shown in FIG. 3. The sliding bar 16 may have a mating V-shape fitting into the shape of the slot 24. This configuration may prevent the sliding bar 16 from slipping out of the bottom 10b of the fracture plate 10. As discussed above, cross links 22 may hold the sliding bar 16 from slipping out of the top 10a of the fracture plate 10.

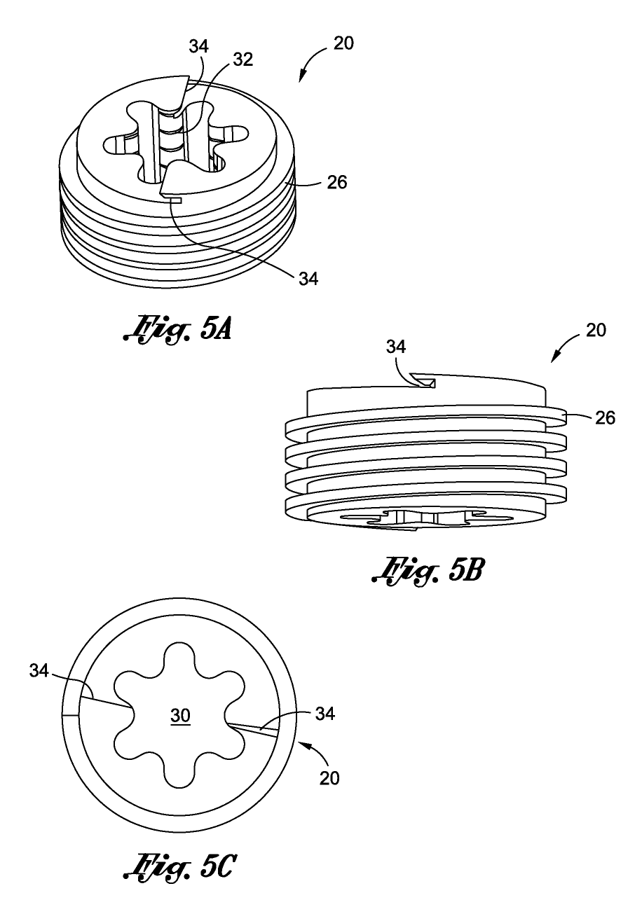

Typically, the set screw 20 may be installed in the fracture plate 10 prior to the fracture plate 10 being implanted across a reduced bone fracture. As shown in FIGS. 4A and 4B, after the fracture plate 10 is implanted (either immediately after implantation prior to closing of the surgical access, or at some later time, as discussed below), the set screw 20 may be removed allowing the sliding plate 16 to move toward the screw holes 12, thereby allowing compression of the fracture. It can be appreciated that, while longitudinal movement (that is, along the length of the bone) of the fracture may occur (which allows for compression of the fracture), rotational and sheer forces may not be imposed on the fracture due to the securing the fracture plate 10 on both sides (via screws (not shown) in screw holes 12 and screw holes 18) of the fracture.

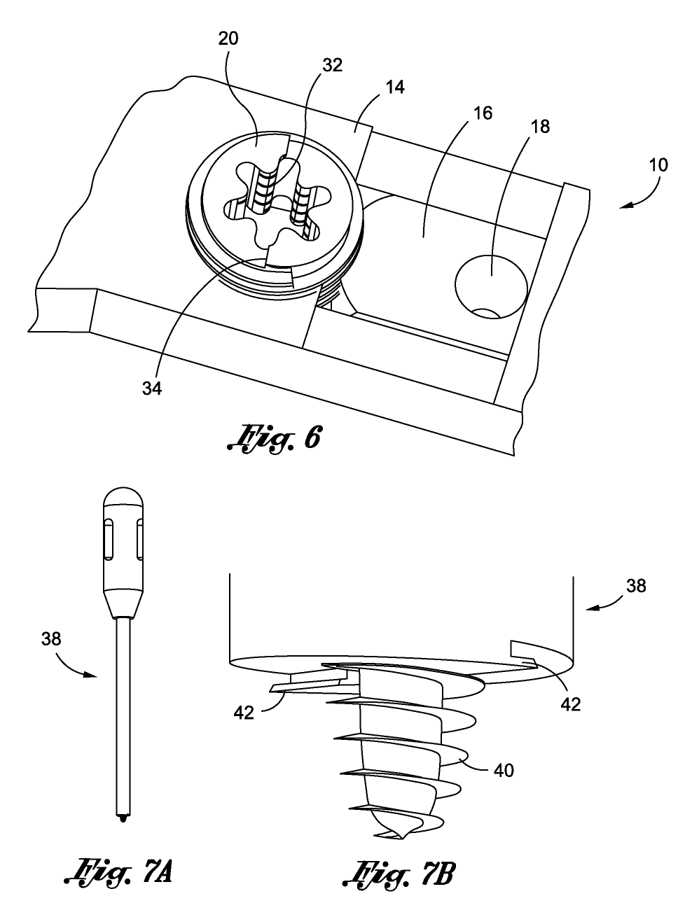

Referring now to FIGS. 5A, 5B and 5C, there are shown perspective, side and top views, respectively, of the set screw 20 (also referred to as locking screw 20) according to the present invention. The set screw 20 may have a standard outer thread 26 that may fit into mating threads 28 (see FIGS. 4A and 4B) of the fracture plate 10. A reverse threaded torx center 30 may include reverse threads 32 for insertion of a screw extractor that is described in greater detail below. The torx center 30 may permit the use of a standard torx implement (not shown) for screw removal if the screw extractor (described below) is not available.

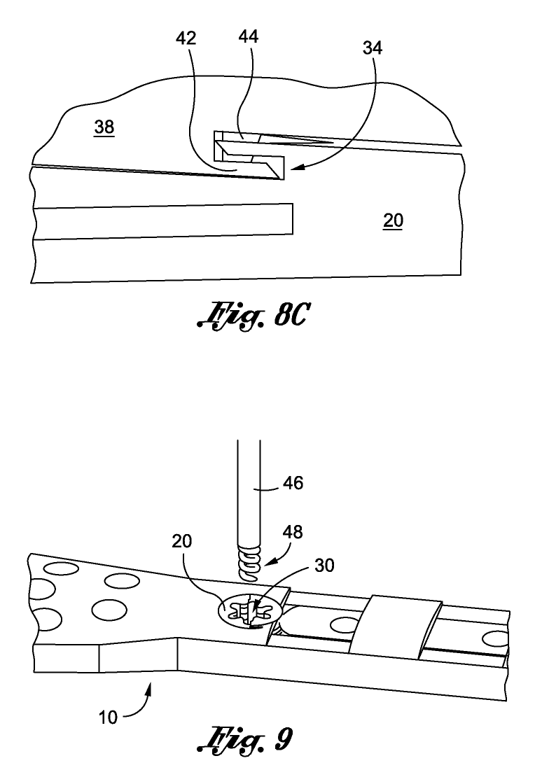

Slots 34 may be cut on a top surface 36 of the set screw 20. When the screw extractor, described below, is reversibly threaded into the threads 32, a portion of the screw extractor may engage the slots 34. See FIGS. 8A-8C below for additional details of this engagement.

Referring to FIG. 6, there is shown a close-up view of the set screw 20 installed into the fracture plate 10. The set screw 20 may hold the sliding plate 16 in a fixed, non-sliding position.

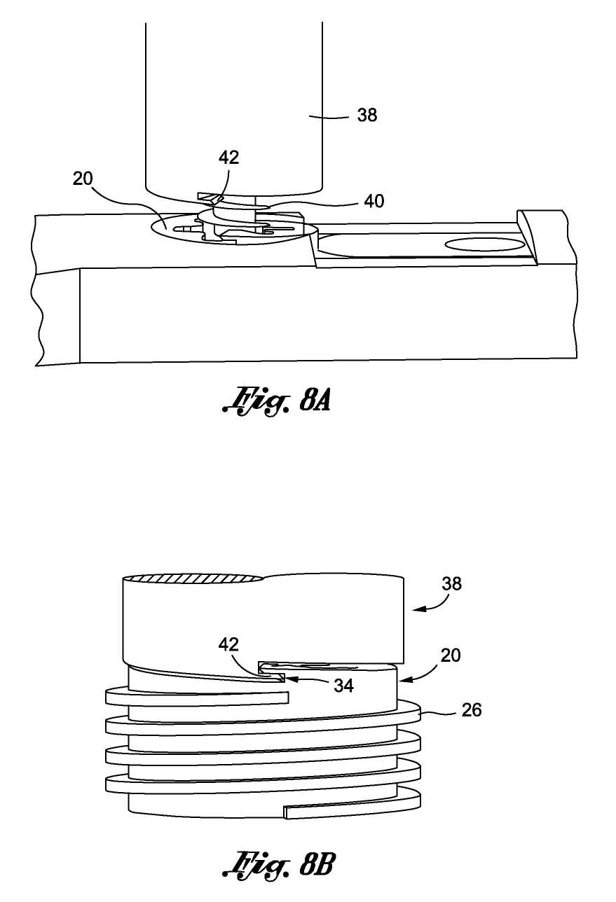

Referring now to FIGS. 7A and 7B, there are shown perspective views of a screw extractor 38 according to the present invention. The screw extractor 38 may have threads 40 which mate to the reverse threads 32 of the set screw 20. When the screw extractor 38 is threaded on the reverse threads 32 in the torx center 30 of the set screw 20, tabs 42 may engage into the slots 34 of the set screw 20. This engagement process is shown in FIGS. 8A through 8C. The slots 34 being engaged by the tabs 42 may help the set screw 20 to be firmly attached to the screw extractor 38 as the extractor 38/set screw 20 combination is extracted through the soft tissues after the set screw 20 is removed from the fracture plate 10.

Referring specifically to FIG. 8C, there is shown a close-up view of the tabs 42 of the screw extractor 38 engaging the slots 34 of the set screw 20. In one embodiment of the present invention, the slots 34 may be made larger than the tabs 42. During engagement of the screw extractor 38 with the set screw 20, a space 44 may be formed therebetween. This space 44 may allow scar tissue (not shown) to be pushed thereinto during the extraction of the set screw 20 from the fracture plate 10.

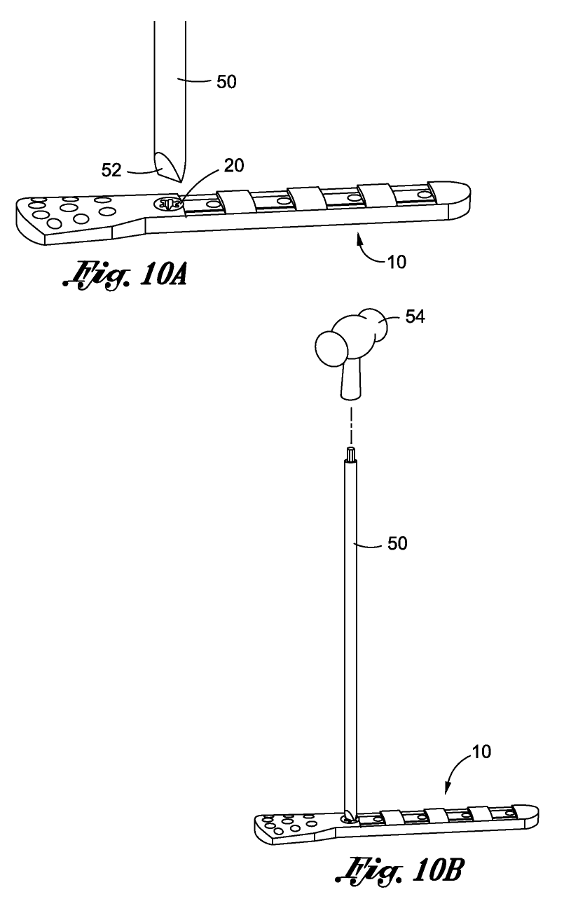

Referring to FIG. 9, while the space 44, described above, allows a location for scar tissue to remain during extraction of the set screw 20, a scar extractor 46 may be used to remove scar tissue (not shown) from the set screw 20 (and, more specifically, from inside the torx center 30 of the set screw 20). A tip 48 of the scar extractor 46 may have a cork screw-like shape for gathering and holding scar tissue as it is removed from around and inside of the set screw 20.

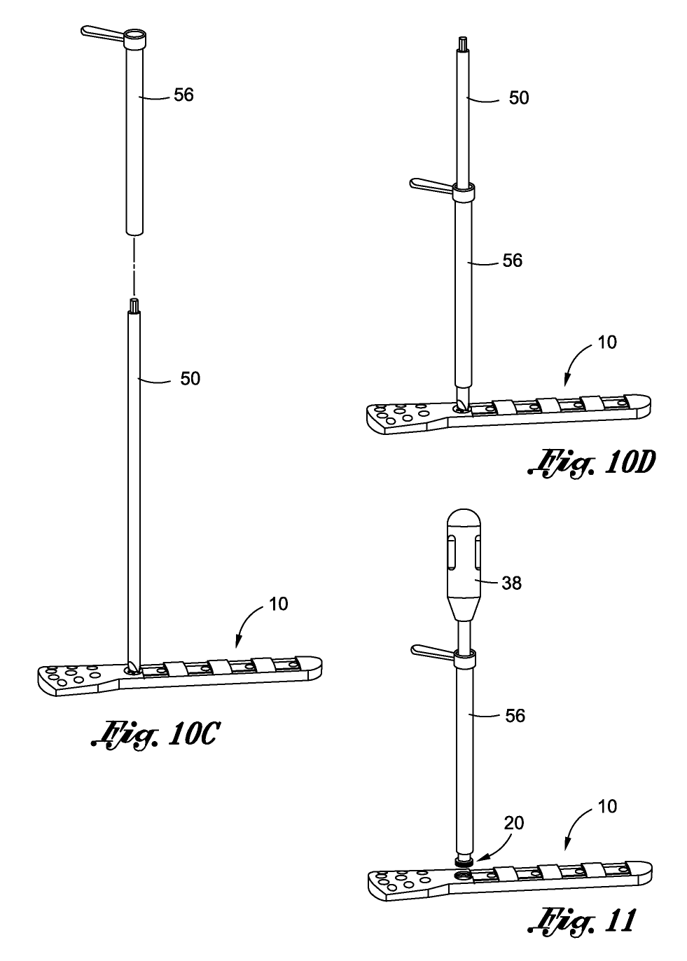

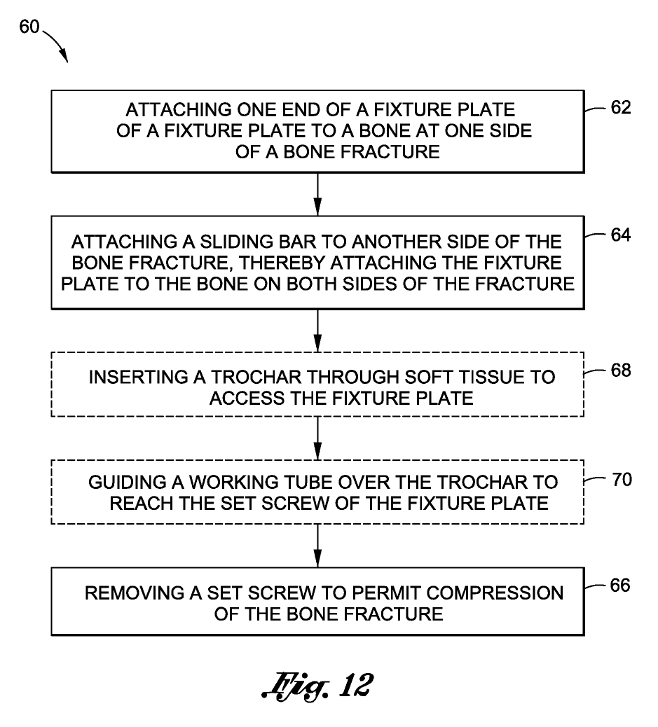

Referring to FIGS. 10A and 10B, if removal of the set screw 20 is desired some time after surgical closure of the fracture, a trochar 50 may be used to gain access to the set screw 20. The trochar 50 may have a tip 52 shaped like a chisel for guiding the trochar 50 through the soft tissue (not shown) to gain access to the set screw 20. Furthermore, the chisel-like tip 52 may also be useful for scraping off soft tissues form the area of the fracture plate 10 near the set screw 20. A handle 54 of the trochar 50 may be removable to allow a working tube 56 to be slid down the trochar 50 and guided to the fracture plate 10, as shown in FIGS. 10C and 10D. The trochar 50 may then be removed and other implements, such as the scar extractor 46 and the screw extractor 38 may be used by insertion through the working tube 56. For example, FIG. 11 shows the screw extractor 38 being guided to the set screw 20 through the working tube 56.

Referring to FIG. 12, there is shown a flow chart describing a method 60 for fixing a fractured bone. The method 60 may include a step 62 of attaching one end of a fracture plate body (e.g., fracture plate body 14) of a fracture plate (e.g., fracture plate 10) to a bone at one side of a bone fracture. The fracture plate may be of the design of the fracture plate described above in accordance with the present invention. The method 60 may also include a step 64 of attaching a sliding bar (e.g., sliding bar 16) to another side of the bone fracture, wherein the fracture plate is attached to the bone on both sides of the bone fracture. The method 60 may further include a step 66 of removing a set screw (e.g., set screw 20) to permit compression of the bone fracture.

The method 60 may contain several optional steps, as shown by dotted lines in the flow chart of FIG. 12. One optional step may include, when the set screw is removed some time after surgical implantation of the fracture plate, a step 68 of inserting a trochar (e.g., trochar 50) through soft tissue to access the fracture plate. A step 70 may include guiding a working tube (e.g., working tube 56) over the trochar to reach the set screw of the fracture plate. The trochar may be removed back through the working tube to allow other implements, such as a scar extractor or a screw extractor to access the set screw.

It should be understood, of course, that the foregoing relates to exemplary embodiments of the invention and that modifications may be made without departing from the spirit and scope of the invention as set forth in the following claims.

I claim:

1. A fracture plate comprising:
a fracture plate body having at least one screw hole therethrough;
a slot formed in the fracture plate body;
a sliding bar slidably disposed in the slot, the sliding bar having at least one screw hole therethrough; and
a set screw threadably disposed in the fracture plate, wherein
the set screw fixes the sliding bar in a first position when the set screw is inserted in the fracture plate; and
the removal of the set screw permits the sliding bar to slide in the slot of the fracture plate.

2. The fracture plate of claim 1, wherein the screw hole in the fracture plate body is formed in one end of the fracture plate body and the slot is formed in a second, opposite end of the fracture plate body.

3. The fracture plate of claim 1, further comprising at least one cross-link disposed across the slot on at least one of the top and the bottom of the fracture plate body, the cross-link securing the sliding bar within the slot.

4. The fracture plate of claim 3, further comprising at least one cross-link disposed across the slot on the top of the fracture plate body and at least one cross-link disposed across the slot on the bottom of the fracture plate body.

5. The fracture plate of claim 1, wherein:
the slot has a V-shaped cross-section and the sliding bar has a mating V-shaped cross-section; and
at least one cross-link is disposed across the slot on the top of the fracture plate body.

6. The fracture plate of claim 1, wherein the slot extends beyond the set screw thereby allowing the sliding bar to slide a distance greater than the diameter of the set screw when the set screw is removed.

7. The fracture plate of claim 1, further comprising reverse threads in the center of the set screw.

8. The fracture plate of claim 7, wherein the center of the set screw is a torx-shaped center.

9. The fracture plate of claim 1, further comprising slots on a top surface of the set screw.

10. A fracture fixation kit comprising:
a fracture plate comprising:
a fracture plate body with at least one screw hole therethrough;
a slot formed in the fracture plate body;
a sliding bar slidably disposed in the slot, the sliding bar having at least one screw hole therethrough; and
a set screw threadably disposed in the fracture plate; and
a screw extractor.

11. The fracture fixation kit of claim 10, further comprising reverse threads in the center of the set screw.

12. The fracture fixation kit of claim 11, wherein a tip of the screw extractor has threads mating with the reverse threads of the set screw.

13. The fracture fixation kit of claim 12, further comprising slots on a top surface of the set screw.

14. The fracture fixation kit of claim 13, further comprising tabs on the screw extractor, the tabs mating with the slots of the set screw when the tip of the screw extractor is threaded into the reverse threads of the set screw.

15. The fracture fixation kit of claim 14, wherein a space is created between the tabs and the slots when the tabs of the screw extractor are mated with the slots of the set screw, the space providing a location for scar tissue to be pushed thereinto when the screw extractor is threaded into the set screw.

16. The fracture fixation kit of claim 10, further comprising a trochar for guiding and introducing a working tube through soft tissue to reach the set screw of the fracture plate.

17. The fracture fixation kit of claim 16, wherein:
the trochar is removable through the working tube when the working tube is guided and introduced to the set screw; and
the working tube allows for the screw extractor to be inserted therethrough.

18. The fracture fixation kit of claim 10, further comprising a scar extractor having a cork screw-shaped tip for removal of scar tissue from the center of the set screw.

19. A method for fixing a fractured bone, the method comprising:
attaching one end of a fracture plate body of a fracture plate to a bone at one side of a bone fracture, the fracture plate comprising:
a slot formed in the fracture plate body at another end of the fracture plate body;
a sliding bar slidably disposed in the slot, the sliding bar having at least one screw hole therethrough; and
a set screw threadably disposed in the fracture plate, the set screw holding the sliding bar at a first position in the slot;
attaching the sliding bar to another side of the bone fracture, wherein the fracture plate is attached to the bone on both sides of the bone fracture; and
removing the set screw to permit compression of the bone fracture.

20. The method of claim 19, further comprising inserting a trochar through soft tissue to access the fracture plate.

21. The method of claim 20, further comprising guiding a working tube over the trochar to reach the set screw of the fracture plate.

22. The method of claim 20, further comprising removing the trochar through a working tube.

23. The method of claim 19, further comprising inserting a scar extractor through a working tube to remove scar tissue from inside and around the set screw.

24. The method of claim 19, further comprising:
inserting a screw extractor through a working tube;
threadably inserting a tip of the screw extractor into mating threads in the center of the set screw; and
securing tabs on the screw extractor into slots on a top surface of the set screw.

25. The method of claim 21, further comprising, after removal of the set screw from the fracture plate, removing the working tube from the fracture plate.
